(19) United States
(12) Patent Application Publication
(10) Pub. No.: US 2006/0178571 A1
(43) Pub. Date: Aug. 10, 2006

(54) Apparatus and method for determining organ perfusion

(75) Inventor: Nicholas James Barnett, Oxford (GB)

Correspondence Address: MERCHANT & GOULD PC, P.O. BOX 2903, MINNEAPOLIS, MN, 55402-0903, US

(73) Assignee: Diametrics Medical Limited, Bucks (GB)

(21) Appl. No.: 10/479,144

(22) PCT Filed: May 31, 2002
(86) PCT No.: PCT/GB02/02546

§ 371 Date: Apr. 27, 2004

(30) Foreign Application Priority Data
Jun. 1, 2001 (GB) 0113388.3

(51) Int. Cl. A61B 5/00; A61M 27/00

(52) U.S. Cl. 600/311; 604/544; 600/549

(57) ABSTRACT

An apparatus for monitoring an analyte comprises a urethral catheter (1), which has positioning means (13) for positioning the catheter within the urethra, and a sensor (19). In use the sensor (19) is held in close proximity to a urethral wall U, permitting monitoring of the analyte level in the urethral wall thereby providing information which may be useful in evaluation of the vita conditions of a patient.

This invention relates to an apparatus and a method for determining analyte perfusion in human and animal organs.

Measurements of oxygen and carbon dioxide tension within various organs in the body have been proposed as an indicator of the onset of shock or sepsis. For example, measurements have been made for this purpose of CO₂ levels within the gut and a correlation has been claimed between such measurements and dysfunction of vital organs, see, for example, U.S. Pat. No. 5,456,251 (Fiddian-Green).

Oxygen partial pressure has also been measured within the bladder and the results indicate a correlation between reduction in the bladder pO₂, lowered blood pressure (BP), reduced aortic blood flow (ABF) and renal blood flow (RBF). A method and apparatus for performing bladder oxygen monitoring for such purpose is described in U.S. Pat. No. 5,389,217 (Singer). Both of the above procedures are based on the premise that, when the body suffers major perfusion failure, e.g. as a result of trauma, infection, cardiac or obstructive shock, blood flow to the vital organs such as the brain, lungs, heart etc is increased, while blood flow to the less important organs is reduced. In the blood flow to non-vital organs this is manifested by a reduction in oxygen and an increase in acidosis and provides an early indication of the onset of critical conditions giving more warning to an intensive care team of the need to take emergency measures to stabilise a patient.

Measurement of CO₂ in the gut has the disadvantage that introduction of a sensor into such a position is generally invasive and requires a separate and complex procedure to install it in the correct position. While the procedure described in the Singer US patent for measurement of oxygen partial pressure in the bladder has yielded impressive results, the particular system described necessarily involves some difficulty in precise location of the sensor in contact with the wall of the bladder.

The present invention is directed to a method and apparatus for determining the onset of shock in a patient by monitoring at least one analyte, for example, by monitoring pCO₂ and/or pO₂ and/or pH, in a manner which overcomes or mitigates the problems described above.

It has now been found in tests on animal models that a sensor placed against the wall of the urethra responds in a similar and simultaneous way to sensors placed against the bladder wall, oesophageal, subcutaneous and skeletal tissue. An in vitro study has shown that the contractile response of the urethra to ischemic injury is more sensitive than that of the bladder, offering a possible explanation for the development and symptoms of urinary incontinence secondary to sphincteric damage before bladder dysfunction is present. (Bratslavsky G et al (2001), J. Urol. 2001:165(6):2086-90). Bratslavsky et al attribute the observed difference in sensitivity of the bladder and urethra to differences in bladder and urethra innervation.

In accordance with one aspect of the present invention, concentration of an analyte, for example, hydrogen ions (pH), oxygen or carbon dioxide, in the epithelial tissue of the urethra is measured by placing a sensor within the urethra. That may be achieved by incorporating a sensor in a urethral catheter so that, when the catheter is located in the normal way for draining urine from the bladder, the sensor is positioned in close proximity to the urethral wall by, for example, incorporating it within an appropriate section of the catheter. By positioning the sensor at or on the surface of the catheter or within a section which is capable of transmitting the analyte, the concentration within the epithelial tissue can be measured. For the purposes of this invention, the term “analyte” includes oxygen, carbon dioxide and hydrogen ions (pH).

The invention provides an apparatus for monitoring an analyte comprising a urethral catheter, the urethral catheter comprising positioning means for positioning the catheter in the urethra, and a sensor which is so located that, when the catheter is positioned within the urethra by the positioning means, the sensor is held in close proximity to a urethral wall.

The apparatus is so arranged that in use the sensor is held in close proximity to a urethral wall. The sensor may be incorporated within a void in the catheter, a portion of the catheter wall being capable of transmitting the analyte, the concentration of which is to be measured. In use, that portion of the wall of the catheter will be placed against the urethral wall.

In the context of this specification, the term “in close proximity to” referring to the relative positioning of the sensor and the urethral wall is to be understood as including direct contact and also including indirect contact in the sense that there may be an intervening medium between the sensor and the urethral wall provided that the intervening medium is capable of transporting the analyte from the urethral wall to the sensor. It may be preferred for the sensor to be separated from the urethral wall, the separation distance and intervening medium being such that a material amount of analyte is able to reach the sensor. It will be appreciated that “a material amount” of analyte will in practice be an amount which is sufficient both to be detectable and to permit changes in the analyte concentration to be effectively monitored. Where the sensor is located within the catheter, the catheter wall may constitute at least a part of said intervening medium. In practice, however, there will generally also be a small volume of fluid between the urethral wall and the sensor. That fluid may be a secretion from the urethral wall. It may, however, be found expedient to deliver a small volume of suitable fluid, for example physiological saline solution, via the catheter.

It is a particular advantage of the apparatus of the invention that reliable monitoring of analyte in the urethral wall can be achieved, using a non-invasive technique. The monitoring of analyte that has diffused through a catheter wall rendered permeable to the analyte and/or through an intervening medium between the catheter wall and the urethral wall (for example, a thin layer of fluid) is believed to give a reliable indication of variations in the analyte concentration in the urethral wall. It will be appreciated that, based on the known principles of diffusion, it will be desirable to position the sensor close to the urethral wall in order to restrict any difference between the measured concentration of the analyte and the actual concentration in the urethral wall. Advantageously, the sensor is so arranged that at least a sensing surface is outside the catheter wall. In an especially preferred arrangement, the sensor is contained within the catheter when the catheter is introduced into the patient and, when the catheter is in position, the sensor can be advanced along the catheter and outwardly therefrom via a suitably positioned exit port. The sensor may in use be within a void of the catheter. In that case, the sensor may advantageously abut a region of the catheter wall that is capable of transmitting analyte. Preferably, the void also includes a fluid which is capable of transmitting the analyte to be measured, the sensor being at least partly immersed in that fluid.

The positioning means may be any positioning device that is suitable for securing the catheter substantially within the urethra with the distal end of the catheter positioned in the bladder and the sensor (located in or outside the catheter) being positioned within the urethra. Advantageously, the positioning means comprises an inflatable balloon, cuff or similar device. Advantageously, the positioning means, especially an inflatable balloon or cuff, is arranged to fit snugly at the neck of the bladder, anchoring the catheter in place and substantially preventing urine from the bladder passing between the catheter and the wall of the urethra, thus substantially eliminating any undesirable influence of urine contamination on analyte measurement.

In an especially preferred apparatus of the invention, the urethral catheter comprises a sensor device which is capable of monitoring at least pH, pO₂ and pCO₂. Devices capable of measuring all those three analytes are known and used, for example, in the blood analyte monitors sold under the trade name Paratrend (Diametric Medical Inc.). Advantageously, the sensor device also includes a temperature-measuring device, for example, a thermocouple.

Advantageously, the catheter comprises a pH sensor positioned within a void in the catheter. In that case, a portion of catheter wall in the region of the pH sensor may be perforated by one or more apertures. Advantageously, the catheter comprises a pO₂ sensor, an adjacent portion of the catheter wall being of a material permeable to oxygen. Advantageously, the catheter comprises a CO₂ sensor, an adjacent portion of the catheter wall being a of material permeable to carbon dioxide.

Urethral catheters are widely fitted as a matter of course to moderately ill patients, especially after an operation or suffering trauma. The invention thus offers a non-invasive technique for monitoring analyte indicative of the condition of the patient, in many cases without necessitating any additional step for introduction of a monitoring device. Further, urethral catheters are normally a close fit within the urethra so that there is no ambiguity in the position of the sensor in relation to the urethral wall. So long as the sensor is positioned in a portion of the catheter which would lie within the urethra, the sensor is automatically positioned in a known relationship with the urethral wall. This is in contrast to the apparatus described in the above-cited Singer US patent, in which the sensor requires to be carefully positioned for contact with the wall of the bladder.

A further advantage of the apparatus of the invention is that the sensor can be protected by being positioned within or on the urethral catheter and covered with a material which is permeable to the analyte.

The invention also provides an apparatus for measuring at least one analyte selected from pH, oxygen and carbon dioxide in urethra epithelial tissue which comprises an urethral catheter having an inflatable device for securing the distal end within the bladder and a sensor so positioned with respect to the catheter that, in use, the sensor lies proximally of the inflatable device within the urethra and in the region of the urethral wall. Moreover, the invention provides a method of monitoring the vital conditions of a patient, comprising positioning a sensor in the urethra of the patient for measuring a concentration of an analyte in the urethral tissue.

It is known that bladder epithelial oxygen tension (BEOT) shows a strong inverse correlation with vital perfusion conditions of the body. The present invention is predicated on the observation that urethral epithelial O₂ tension and, additionally or instead, urethral epithelial CO₂ tension can be used as an indication of gaseous perfusion in vital organs and therefore give a pre-warning of critical conditions in a patient.

Three illustrative embodiments of the invention will now be described with reference to the accompanying drawings, in which:

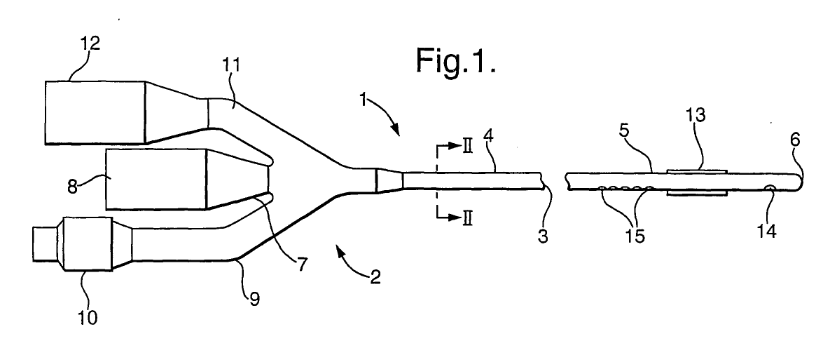

FIG. 1 is a side elevation of an urethral catheter having three lumens;

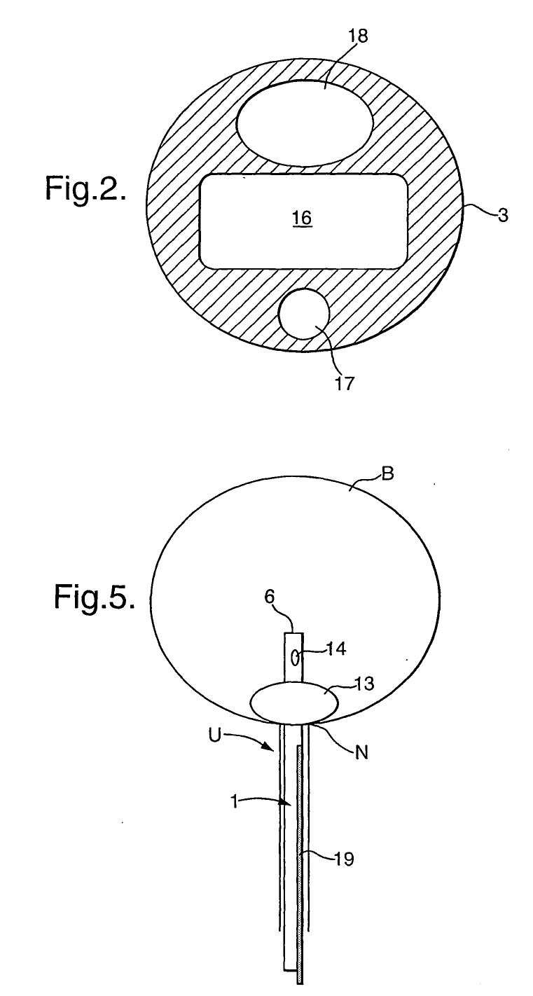

FIG. 2 is a transverse section through a proximal portion of the catheter of FIG. 1;

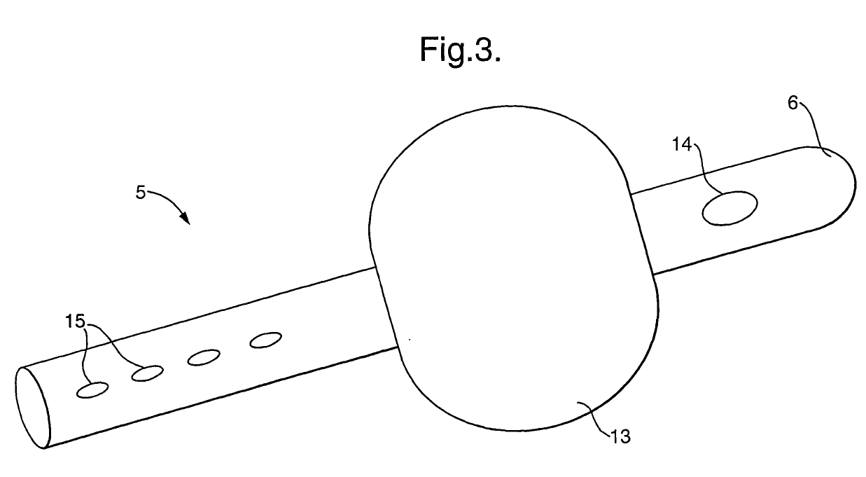

FIG. 3 is an enlarged view from below of a distal portion of the catheter of FIG. 1;

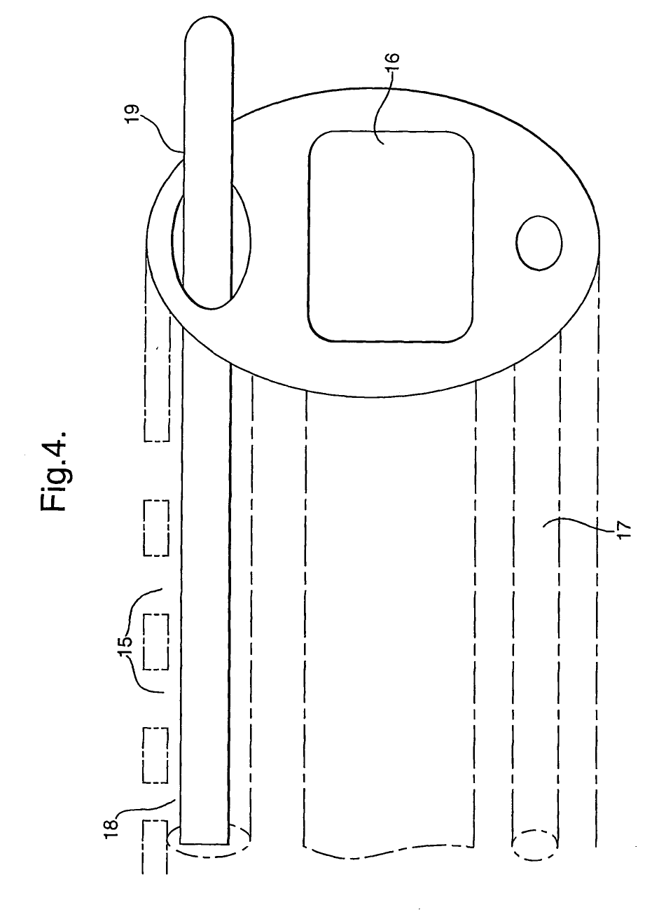

FIG. 4 is a schematic view, partly in section, of a part of the distal portion;

FIG. 5 is a schematic view of the catheter positioned within the urethra of a patient.

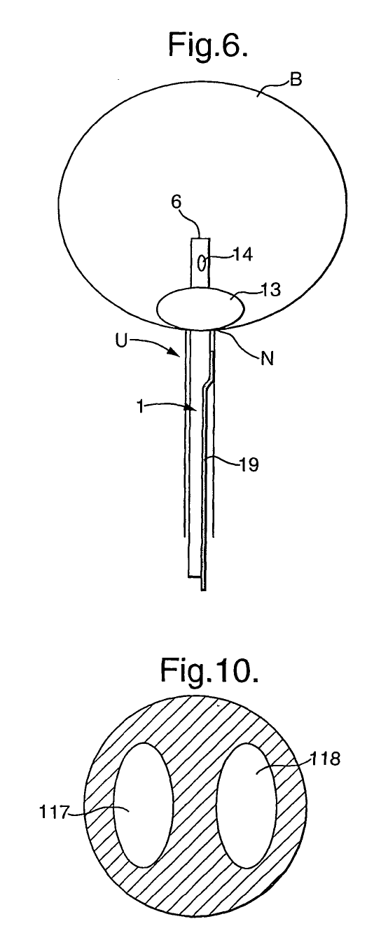

FIG. 6 is another form of three-lumen catheter;

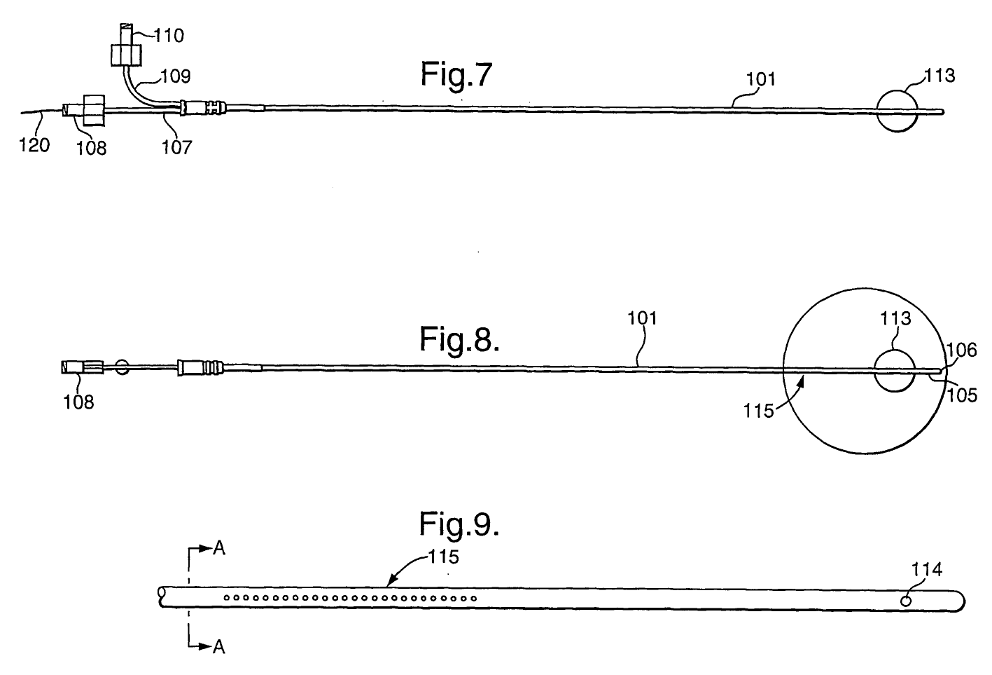

FIG. 7 is a side elevation of a second urethral catheter;

FIG. 8 is a view of the second catheter seen from below;

FIG. 9 is a view on an enlarged scale of the portion of the second catheter circled in FIG. 7; and

FIG. 10 is an enlarged section taken along the line A-A in FIG. 8.

With reference to FIG. 1, a urethral catheter 1 is formed as a three-lumen catheter having a connector portion 2 and an elongate body 3. The elongate body 3 has a proximal portion 4, adjacent to connector portion 2, and distal portion 5 including tip 6. The body 3 has a urine drainage lumen communicating via a cannula 7 with urine drainage channel connector 8, an air lumen communicating via cannula 9 with air lumen connector 10, and a sensor lumen communicating via cannula 11 with sensor lumen connector 12. The urine drainage lumen connector 8 is arranged to be attachable to a suitable urine collection device of any suitable kind. The air lumen connector 10 is arranged to be attachable to a suitable source of air, for example a syringe, for inflation of a catheter positioning device as will be described further below. The sensor lumen connector 12 is arranged for attachment to a sensor monitoring device, which may be for example a device capable of generating light and/or other signals to one or more sensor devices and of receiving information fed back from said sensor(s). Thus, the sensor lumen connector 12 may include a connection device for connecting one or more optical fibres.

The distal portion 5 of the catheter is provided with an inflatable balloon 13, which is shown deflated in FIG. 1. Distally of the balloon 13, and adjacent to the tip 6, is a lateral urine drainage aperture 14. Proximally of the balloon 13, is provided a longitudinally extending row of apertures 15. The apertures 15 may suitably be of about 0.4 mm diameter at equal pitches of 1 mm (the drawing of FIG. 1 is not to scale). Other sizes and/or patterns of apertures may, however, be used. Where the analyte to be measured is hydrogen ion concentration, the apertures 15 are filled with a hydrophilic substance which provides a pathway for water-borne hydrogen ions to pass into the catheter. Suitable hydrophilic substances include hydrophilic gels, for example, a polyacrylamide gel.

Referring now to FIG. 2, the elongate body 3 defines three longitudinally extending lumens. Urine drainage channel 16 extends between drainage aperture 14 and urine drainage lumen connector 8 for drainage of urine from the aperture 14. Air lumen 17 extends from the air lumen connector 10 to the inflatable balloon 13 for the introduction of air to inflate the balloon 13 when required. A third lumen, sensor lumen 18, extends from the sensor lumen connector 12 along the elongate body 3 into the distal portion 5 and is closed at or in the vicinity of the balloon 13. The apertures 15 in the wall of the catheter are arranged to be in communication with the sensor lumen 18 in order to permit fluid carrying an analyte to flow into the sensor lumen 18 when the wall of the catheter is, in use, pressed into intimate contact with the wall of the urethra. The cross-sectional shape of each lumen may vary from that shown, although in the case of the sensor lumen it may be preferred for at least the outermost surface to be substantially parallel to the outer catheter wall in at least the region of the apertures 15.

The distal portion 5 of the catheter 1 is shown in enlarged view in FIG. 3, in which the balloon 13 is shown inflated.

Referring to FIG. 4, a sensor 19 is located within sensor lumen 18 adjacent to the row of apertures 15 for receiving fluid which flows there through.

Additionally, or as an alternative to forming apertures in the wall of the catheter, the wall in the region in which in use the sensor is located may be made from a material, for example, a membrane, which is permeable to an analyte gas of interest. For example, a membrane made from a silicone rubber will be permeable to CO₂. The sensor may be of the optical type used to measure oxygen or carbon dioxide partial pressure or, alternatively, of the electrochemical type. Both types of sensors are available from Diametrics Medical Limited and are included in pCO₂ and PO₂ monitoring equipment marketed under the trade marks “Continucath” and “Paratrend”. Preferably, the sensors used are of the optical type since these can be made as solid state sensors which do not consume chemicals. The preferred type of sensors include a chemical indicator which is sensitive to the gaseous analyte and undergoes a colour change or fluorescent properties depending on the concentration of the analyte. The chemical indicator is preferably a fluorescent dye which is quenched by the gaseous analyte. Optical fibres (not shown in FIG. 1 but shown in FIG. 7) are employed for conducting light of the wavelength at which the dye fluoresces and for conducting the returning beam to a detector remote from the catheter. The intensity of the fluorescence can be used as a continuous measure of the concentration of the analyte, after any necessary calibration of the sensor.

In the case of some sensors, particularly pH sensors, it may be desirable to incorporate a reservoir of saline or water around the sensor within the sensor lumen 18. This is to ensure that hydrogen ions can diffuse into contact with the sensor.

FIG. 5 shows the distal portion of the catheter in position in a patient. In use, the catheter 1 will be introduced along the urethra into the bladder in the usual way for installing a urethral catheter so that the tip 6 lies within the patient's bladder B and liquid may be drained through the drainage lumen 16. On inflation of the balloon 13 at the neck N of the bladder B, the distal tip 6 is held clear from the walls of the bladder B and a major portion of the catheter which lies proximally of the balloon 13 will be within the urethra U.

A second form of urethral catheter according to the invention is shown in FIG. 6. The catheter of FIG. 6 is essentially the same as that of FIGS. 1 to 5 except that the sensor device is arranged to protrude outwardly, in use, from the sensor lumen and to lie between the catheter and the urethral wall. It will be appreciated, therefore, that the provision of the apertures 15 of the embodiment of FIGS. 1 to 5 is unnecessary and that, instead, there is provided in the wall of the catheter an aperture through which the sensor can be advanced once the catheter is in place in the urethra.

The present invention has the advantage that by ensuring that the catheter is positioned with the inflatable device such as a balloon or cuff anchoring the catheter in the neck of the urethra, the location of the sensor is known with certainty and consistent measurements can be made of the analyte perfusion concentration within the urethral epithelial tissue. Furthermore those measurements would be expected to give a good correlation with analyte perfusion concentration within the bladder epithelial tissue, whilst offering the above-described simplicity and consistency of location, and to critical conditions a possibility of slightly improved tissue responsiveness as compared with the bladder. The invention also offers the possibility of essentially continuous monitoring of the analyte or analytes, providing the clinician with an effectively instantaneous measurement of the current value as well as an indication of the historical development of the analyte level.

Urethral catheters of the general type described above which are anchored into a patient's bladder using an inflatable device are commercially available as Foley catheters. The catheters of the invention, including a sensor, could therefore be regarded as modified Foley catheters. Foley catheters are conventionally fitted to moderately ill patients, especially after an operation or suffering trauma, e.g. as a result of haemorrhage. The catheter can be introduced using conventional procedures so that the installation of the catheter does not involve any novel steps or manipulations. The only additional requirement is that the cable or optical fibres which are connected to the sensor will project, e.g. from the connector 12 and require connection to a monitor. The familiar Luer type locks can be used for this purpose. This is a clear advantage since when nursing critically ill patients, it is desirable to reduce the number of additional connections and manipulations which need to be made in the critical care environment.

The positioning of the catheter is reliably reproducible, the position being essentially governed by the air-filled balloon which, in practice, is arranged to fit snugly at the neck of the bladder at the entrance to the urethra. Thus, once the balloon is inflated in the correct position monitoring takes place at a fixed distance proximal to the neck of the bladder.

Referring to FIG. 7, a second form of urethral catheter 101 is provided which is a bi-lumen catheter having a first lumen 117 for inflating a locating balloon 113 (shown inflated in FIGS. 7 and 8, but omitted for clarity in FIG. 9). Lumen 117 is connected with a cannula 109 and terminates in a connector 110 to which it may be connected to means for inflating the balloon such as a syringe.

Lumen 116 is connected at the proximal end with a cannula 107 which is linked to a connector 108 for connection to tubes for draining urine from the bladder. In use, the catheter 101 will be introduced along the urethra into the bladder in the usual way for installing a urethral catheter so that the distal end 106 lies within the patient's bladder and liquid may be drained through the drainage lumen 116. On inflation of the balloon 13, the distal end is held clear from the walls of the bladder and a major portion of the catheter which lies proximally of the balloon 113 will be within the urethra. The side wall of the catheter is perforated over a section 115 which lies proximally of the balloon 113, the perforations extending into the drainage lumen 116.

As shown in FIG. 9, a pattern of aligned holes 115 of about 0.4 mm diameter are provided at equal pitches of 1 mm to permit fluid to flow into the drainage lumen 116 while the wall of the catheter is pressed into intimate contact with the wall of the urethra. It will be appreciated, however, that other sizes and patterns of holes can be provided into the drainage lumen 116. The holes may be filled with a hydrophilic material, for example, a polyacrylamide gel. A further hole or holes 114 is formed in the distal portion 105 of the catheter distally of balloon 113. The hole 114 also communicates with the drainage lumen 116 to drain urine from the bladder through the cannula 107 away from the patient.

A sensor (not shown) is positioned within the drainage lumen 116 in the vicinity of the perforated section 115 so that the sensor is exposed to gases present in tissue in the urethral wall. Communication means, for example one or more cables or optical fibres 120, for signals from the sensor extend individually along the catheter within the drainage lumen 116 from the sensor to the connector 108, which is arranged for connection to a sensor monitoring device. As described above with reference to FIGS. 1 to 5, as an alternative to forming apertures in the wall of the catheter, the wall at this point may be made from a material which is permeable to the analyte gas of interest. In practice, however, it will be preferred in the case of the two-lumen catheter of FIGS. 7 to 10 for the sensor to be so arranged that it protrudes outside the catheter, and may then contact directly the urethral wall, thus avoiding the influence on the measurements of urine passing through the drainage lumen. In that case, the aligned holes 115 may be absent and may be replaced by an aperture which is positioned and dimensioned to permit at least a sensing portion of the sensor to be advanced therethrough and located in analogous manner to that shown in FIG. 6 once the catheter has been positioned in the urethra. Suitable sensor devices for use in the catheter of FIGS. 7 to 10 are, for example, those described above with reference to FIGS. 1 to 5.

1.-20. (canceled)

21. An apparatus for monitoring an analyte comprising:
(a) a urethral catheter; the urethral catheter including:
(i) a positioning arrangement for positioning the catheter in the urethra, and
(ii) at least one sensor oriented such that when the catheter is positioned within the urethra by the positioning arrangement, the at least one sensor is held in close proximity to a urethral wall.

22. An apparatus according to claim 21, wherein:
(a) the catheter includes a distal end; and
(b) the positioning arrangement comprises an expansible device arranged for holding the distal end of the catheter in the bladder in use; and the at least one sensor is located proximally of the expansible device so that the at least one sensor lies within the urethra.

23. An apparatus according to claim 21 wherein:
(a) the positioning arrangement comprises an inflatable balloon.

24. An apparatus according to claim 21 wherein:
(a) the at least one sensor is capable of measuring at least one analyte selected from pH, pO₂ and pCO₂.

25. An apparatus according to claim 21 wherein:
(a) the at least one sensor is capable of measuring at least one analyte through optical detection.

26. An apparatus according to claim 24 wherein:
(a) the at least one sensor includes at least two sensors, each of the at least two sensors being capable of measuring a respective different analyte; a respective sensor being present for measuring the respective different analyte.

27. An apparatus according to claim 21 wherein:
(a) the at least one sensor is positioned within the catheter; and
(b) a region of the catheter wall near the at least one sensor is capable of transmitting an analyte to be measured by the at least one sensor.

28. An apparatus according to claim 27 wherein:
(a) the at least one sensor is a pH sensor positioned within the catheter; and
(b) a region of the catheter wall is perforated or is porous to hydrogen ions.

29. An apparatus according to claim 27 wherein:
(a) the at least one sensor is a sensor for dissolved carbon dioxide located within the catheter; and
(b) at least a region of the catheter wall is permeable to carbon dioxide.

30. An apparatus according to claim 27 wherein:
(a) the at least one sensor is a sensor for dissolved oxygen is located within the catheter; and
(b) at least a region of the catheter wall is permeable to oxygen.

31. An apparatus according to claim 30 wherein:
(a) the sensor is an optical device in which a fluorescent material is quenched by the analyte.

32. An apparatus according to claim 21 wherein:
(a) the at least one sensor includes a common sensor device capable of measuring pO₂ and pCO₂ and pH.

33. An apparatus according to claim 21 further comprising:
(a) a temperature sensing device for measuring temperature.

34. An apparatus according to claim 21 wherein:
(a) the urethral catheter is a triple lumen catheter comprising:
(i) a first lumen for inflating a positioning arrangement;
(ii) a second lumen for draining urine; and
(iii) a third lumen for housing the at least one sensor in close proximity to the urethral wall.

35. An apparatus for measuring at least one analyte selected from pH, oxygen and carbon dioxide in urethra epithelial tissue, the apparatus comprising:
(a) an urethral catheter including:
(i) an inflatable device to secure a distal end of the catheter within a bladder; and
(ii) a sensor positioned with respect to the catheter to lie proximally of the inflatable device within the urethra and in a region of an urethral wall, when in use.

36. An apparatus according to claim 35 wherein:
(a) the urethral catheter is a triple lumen catheter comprising:
(i) a first lumen for inflating the inflatable device,
(ii) a second lumen for draining urine; and
(iii) a third lumen for housing the sensor in close proximity to the urethral wall;
(A) the sensor including at least one or more sensors; each of the one or more sensors being housed by the third lumen.

37. A method of monitoring vital conditions of a patient, the method comprising:
(a) positioning a sensor in a urethra of the patient for measuring a concentration of an analyte in urethral tissue.

38. A method according to claim 37 wherein:
(a) the step of positioning includes introducing the sensor to the urethra with a catheter.

39. A method according to claim 38 further including:
(a) draining urine from the patient through a lumen of the catheter; and
(b) providing a closure distally of the sensor to prevent passage of urine into the urethra other than within the lumen.

40. A method of monitoring a patient; the method comprising:
(a) introducing into a urethra of a patient a urethral catheter having a sensor for sensing an analyte and a positioning arrangement; and
(b) positioning the catheter so that the sensor is held in close proximity to a urethral wall by using the positioning arrangement.

41. A method according to claim 40 wherein:
(a) the step of introducing includes advancing a distal end of the catheter in to the patient's bladder; and
(b) the step of positioning includes expanding an expansible device located at the distal end of the catheter to retain the distal end within the bladder, with the sensor being located proximally of the expansible device and within the urethra.

42. A method according to claim 40 wherein:
(a) said step of positioning includes inflating an inflatable balloon.

43. A method according to claim 40 wherein:
(a) said step of introducing includes introducing a urethral catheter having a sensor for sensing an analyte selected from pH, pO₂ and pCO₂.

44. A method according to claim 43 wherein:
(a) said step of introducing includes introducing a urethral catheter having a sensor for sensing temperature.

45. A method according to claim 40 further including:
(a) monitoring pH, pO₂ and pCO₂.

46. A method according to claim 40 further including:
(a) monitoring at least one analyte with optical detection.

47. A method according to claim 46 further including:
(a) allowing at least one analyte to diffuse into the sensor and interact with a chemical indicator.

48. A method according to claim 40 further including:
(a) monitoring dissolved oxygen including permitting oxygen to permeate through a region of a wall of the catheter wall to gain access to an oxygen sensor located within the wall.
